(19) United States
(12) Patent Application Publication
(10) Pub. No.: US 2005/0171119 A1
(43) Pub. Date: Aug. 4, 2005

(54) Pharmaceutical formulations with modified release

(75) Inventors: Monique Berwaer, Ham-sur-Heure-Nalinnes (BE); Michel Deleers, Linkebeek (BE); Domenico Fanara, Wanze (BE); Anthony Guichaux, Fribourg (CH)

Correspondence Address: WENDEROTH, LIND & PONACK, L.L.P., 2033 K STREET N. W., SUITE 800, WASHINGTON, DC, 20006-1021, US

(73) Assignee: UCB, S.A., Brussels (BE)

(21) Appl. No.: 10/500,454

(22) PCT Filed: Jan. 8, 2003
(86) PCT No.: PCT/EP03/00093

(30) Foreign Application Priority Data
Jan. 9, 2002 (EP) 02000521.1

(51) Int. Cl. A61K 31/495; A61K 9/22

(52) U.S. Cl. 514/255.04; 424/468

(57) ABSTRACT

The invention relates to a pharmaceutical composition which can be administered orally, containing efletirizine as active principle, and combining a fraction which allows immediate release and a fraction which allows prolonged release.

The present invention relates to a modified-release pharmaceutical composition for administering an effective amount of 2-[2-[4-[bis(4-fluorophenyl)methyl]-1-piperazinyl]ethoxy]acetic acid and of its pharmaceutically acceptable salts in order to obtain both rapidly an effective plasma concentration and also maintenance of a minimum effective concentration over a prolonged period. The modified-release composition comprises at least two fractions containing the active principle. The first fraction allows immediate release of the active principle and the second allows prolonged release of the active principle and maintenance of an effective plasma concentration over a prolonged period. The compositions obtained are particularly suitable for administration in a single daily dose.

2-[2-[4-[Bis(4-fluorophenyl)methyl]-1-piperazinyl]-ethoxy]acetic acid, also known as and referred to here as efletirizine (INN: International Nonproprietary Name), is the compound of formula below:

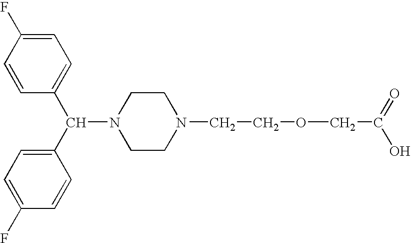

Efletirizine is encompassed in general formula I of European patent EP-B-0 058 146 in the applicant's name, which relates to substituted benzhydrylpiperazine derivatives.

Like 2-[2-[4-[(4-chlorophenyl)phenylmethyl]-1-piperazinyl)ethoxy]acetic acid, also known as and referred to herein as cetirizine, it has been noted that efletirizine has excellent antihistamine properties. It belongs to the pharmacological category of second-generation histamine H₁ receptor inhibitors and exhibits great affinity and great selectivity in vitro for H₁ receptors. Like cetirizine, it is useful as an antiallergic agent, an antihistamine, a bronchodilator and an antispasmodic. Recent clinical studies have shown the usefulness of efletirizine when it is administered in the form of a nasal spray for the treatment of allergic rhinitis and rhinoconjunctivitis (J. F. Dessanges et al., Allergy et Clin. Immunol. News (1994), Suppl. No. 2, Abstract 1864; C. De Vos et al., Allergy et Clin. Immunol. News (1994), Suppl. No. 2, Abstract 428). Another recent clinical pharmacology study has shown that efletirizine gives surprisingly good results in the treatment of urticaria, atopic dermatitis and pruritis. In European patent EP-B-1 034 171 in the applicant's name, two pseudo-polymorphic crystalline forms of efletirizine dihydrochloride, namely anhydrous efletirizine dihydrochloride and efletirizine dihydrochloride monohydrate, were discovered and described for their particularly advantageous physical properties.

International patent application WO 98/41194 describes a pharmaceutical composition which can be administered orally, and which comprises at least one layer comprising an active substance and excipients which allow the immediate release of said active substance after administration, and at least a second layer which allows the controlled release of the same or of a second active substance, comprising said same or second active substance, at least one excipient of matricial type, and at least one basifying agent. The composition comprises between 5 and 50% by weight, relative to the total weight of the composition, of basifying agent soluble in an aqueous phase under physiological pH conditions. Because of the presence of the basifying agent, this composition has shown a good stability profile.

Due to an increasing therapeutic interest in efletirizine, the preparation of novel pharmaceutical compositions containing this active principle has been undertaken.

In order to facilitate treatment of the patient, it is desirable to develop novel pharmaceutical compositions which can be administered orally and to control the release of the pharmaceutically active substances in such a way that they may be administered in a single daily dose while at the same time exhibiting a rapid therapeutic activity.

It is known that the bioavailability of certain active principles can be modified by means of a prolonged-release formulation which releases the active principle gradually over the entire length of the gastrointestinal tract, over a longer period of time, and thus avoids repeated absorption of a pharmaceutical composition by the patient.

It has, however, been established that such prolonged-release pharmaceutical formulations commonly used by those skilled in the art and applied to efletirizine have the disadvantage of reaching the effective plasma concentration later and therefore of delaying the therapeutic action of the active principle. In addition, it has been noted that such prolonged-release formulations, compared to the administration of two immediate-release doses 12 hours apart, induce a decrease in the maximum plasma concentration of efletirizine and also a decrease in its bio-availability. In fact, the various studies carried out have also revealed that efletirizine exhibits better absorption in the upper portions of the gastro-intestinal tract. Now, in order to relieve the patient as quickly as possible, it is desirable to rapidly provide him or her with a therapeutically effective dose of efletirizine while at the same time maintaining an effective minimum concentration for as long as possible, and preferably around 24 hours. In addition, all these conditions must be met while maintaining a bioequivalence with two administrations of 5 to 25 mg of efletirizine in an immediate-release form, given 12 hours apart.

In the interests of developing novel pharmaceutical compositions which can be administered orally and which can be administered in a single daily dose, it has been discovered that combining a fraction which allows immediate release of the active principle with a second fraction which allows prolonged release of the active principle makes it possible to ideally satisfy the specific pharmacokinetic requirements related to the use of efletirizine and to a better control of the effect of having a meal. One great difficulty has been to find the correct balance between the immediate release of the active principle and the prolonged release for maintaining an effective dose for a long period of time while avoiding reaching the plasma concentration peak, engendering side effects.

The novel compositions thus developed have also shown, particularly surprisingly, that by combining at least one immediate-release fraction and at least one prolonged-release fraction, the pharmaceutical compositions thus obtained make it possible to reduce, in a more or less substantial manner, according to the distribution of the active principle between the fractions, the influence of having a meal before their absorption by the patient, this effect absolutely not being observed for immediate-release compositions. This unexpected discovery is particularly useful for maintaining the bioequivalence and the maximum plasma concentration of a pharmaceutical composition, whether it is taken before or after the meal, and, as a result, the consequences of incorrect handling or use by the patient are reduced.

Moreover, surprisingly, it has been shown that a composition containing less than 5% of basifying agent, or not containing any, can be prepared and that the absorption is found to be constant and independent of the pH.

Thus, a first aspect of the present invention concerns novel pharmaceutical compositions which can be administered orally, containing efletirizine as active principle, characterized in that they combine at least one fraction which allows immediate release of the active principle and at least one fraction which allows prolonged release of the active principle. The total amount of efletirizine in the composition is between 10 and 70 mg, and the weight ratio of the amount of active principle in the immediate-release fraction to the amount of active principle in the prolonged-release fraction is between 3 and 0.025. The amount of active principle is expressed as amount of efletirizine in its dihydrochloride form.

Such modified-release compositions can be adjusted for administrations of one to three doses per day.

Finally, continuing the search for an ideal composition for limiting the number of daily administrations, specific quantitative ratios of active principle in the immediate-release fraction to active principle in the prolonged-release fraction have been demonstrated in order to satisfy the various abovementioned requirements by being bioequivalent to two administrations of immediate-release forms while at the same time controlling the influence of having a meal. These quantitative ratios make it possible to obtain pharmaceutical compositions which are particularly insensitive to having a meal.

Thus, in a second aspect, the present invention relates to novel pharmaceutical compositions which can be administered orally, containing efletirizine as active principle, characterized in that they combine at least one fraction which allows immediate release of the active principle and at least one fraction which allows prolonged release of the active principle, the respective amounts of active principle in the two fractions being the values included on or between the two straight lines defined by the following equations:
Y=−0.6786X+56.675
Y=−0.6636X+7.975
in which,
Y represents the amount of active principle in mg in the immediate-release fraction, and
X represents the amount of active principle in mg in the prolonged-release fraction,
the total amount of X+Y being between 10 and 70 mg.

The present invention preferably relates to an oral composition which can be administered in a single daily dose.

The content of efletirizine in the composition of the invention is based on the amount of anhydrous efletirizine dihydrochloride. The contents may vary according to the type of efletirizine used (water content, type of salt, form, etc.).

The term “active principle” is intended to mean 2-[2-[4-[bis(4-fluorophenyl)methyl]-1-piperazinyl]-ethoxy]acetic acid (efletirizine) or one of its pharmaceutically acceptable salts.

The term “efletirizine” is intended to mean 2-[2-[4-[bis(4-fluorophenyl)methyl]-1-piperazinyl]-ethoxy]acetic acid or one of its pharmaceutically acceptable salts.

The term “pharmaceutically acceptable salts” is intended to mean not only the addition salts of pharmaceutically acceptable nontoxic acids, such as acetic acid, citric acid, succinic acid, ascorbic acid, hydrochloric acid, hydrobromic acid, sulfuric acid, phosphoric acid, etc., but also the metal salts (for example sodium salts or potassium salts) or the ammonium salts. Efletirizine dihydrochloride is preferably used.

In addition, the various crystalline forms of efletirizine or of its pharmaceutically acceptable salts can be used according to the present invention, for example the anhydrous and hydrated forms of efletirizine dihydrochloride, the pseudopolymorphic crystalline forms of efletirizine dihydrochloride, namely anhydrous efletirizine dihydrochloride and efletirizine dihydrochloride monohydrate.

The expression “fraction which allows immediate release” is intended to mean a pharmaceutical composition which will release the active principle very rapidly after it has been absorbed, and in general in the upper portions of the gastrointestinal tract.

The expression “fraction which allows prolonged release” is intended to mean a pharmaceutical composition which makes it possible to distribute the release of the active principle over a reasonably long period of time compared with a fraction which allows immediate release.

The term “modified-release composition” is intended to mean a composition which combines two different types of releases; in our case, immediate release and prolonged release.

The term “bioavailability” is intended to mean the rate and the amount of active principle absorbed starting from its pharmaceutical form so as to become available at its site of pharmacological action.

Two pharmaceutical compositions are referred to as “bioequivalent” if they are pharmaceutically equivalent or pharmaceutical alternatives and if their respective bioavailability (areas under the curve (AUC) not statistically different) after administration is sufficiently similar to obtain an equivalent therapeutic efficacy.

Immediate-release or prolonged-release pharmaceutical compositions are well known in the literature and should all make it possible to implement the present invention. It is important to emphasize that the beneficial effects of the invention should be observed, whatever the types of immediate-release and prolonged-release fractions that the specialist in the technical field decides to combine.

The release of active substances by the prolonged-release fraction, during oral administration, can be controlled by means of pharmaceutical compositions of matricial type. Three types of matrices: inert, hydrophilic and lipophilic matrices, are distinguished according to the excipients used. By combining excipients of these various types of matrices, it is also possible to create mixed matrices. Many other effective means for controlling the release of active substances are described in the literature, such as, for example, galenic forms with a barrier coating, osmotic forms, floating forms or bioadhesive forms.

Preferably, in the composition according to the invention, the fraction which allows prolonged release of the efletirizine contains less than 5% by weight of basifying agent, relative to the total weight of the fraction.

The inert matrices comprise excipients belonging essentially to the class of thermoplastic polymers. They are inert with respect to the biological tissues, to the other excipients in the formulation and to the active principle. They are insoluble and indigestible in the fluids of the gastrointestinal tract. Among these, mention may be made of polyvinyl chloride, polyethylene, copolymers of vinyl acetate and vinyl chloride, poly(methyl methacrylates), polyamides, silicones, ethylcellulose, polystyrene, etc. They are generally used at a concentration ranging from 20 to 95%.

The hydrophilic matrices comprise gelling excipients divided up into three classes: cellulose derivatives (hydroxypropylmethylcellulose, hydroxyethylcellulose, hydroxypropylcellulose, methylcellulose, etc.), non-cellulose polysaccharides (galactomannans, guar gum, carob gum, gum arabic, sterculia gum, agar agar, alginates, etc.) and acrylic acid polymers (carbopols 934P and 974P, etc.). They are generally used at a concentration of 20 to 70%.

The lipid matrices comprise four types of fatty excipients: glycerides (mono- di- or triglycerides: stearin, palmitin, laurin, myristin, hydrogenated castor or cottonseed oils, precirol, etc.), fatty acids and fatty alcohols (stearic acid, palmitic acid, lauric acid; stearyl alcohol, cetyl alcohol, cetostearyl alcohol, etc.), fatty acid esters (monostearates of propylene glycol and of sucrose, sucrose distearate, etc.) and waxes (white wax, sperm whale wax, etc.). They are generally used at a concentration of 10 to 50%.

As regards the excipients which allow immediate release of the active principle, they can be chosen from diluents (calcium phosphate, lactose, etc.), binders (microcrystalline cellulose, starches, polyvinyl-pyrrolidone, etc.), disintegrating agents (starches and modified starches, cellulose derivatives, alginic derivatives, pectins, etc.), lubricants and flow enhancers (talc, magnesium stearate, colloidal silica, etc.), taste-masking agents (α-cyclodextrin, β-cyclodextrin, γ-cyclodextrin and derivatives thereof), flavorings or colorants.

Besides the abovementioned constituents, the pharmaceutical compositions according to the present invention can also contain other excipients, such as diluents (calcium phosphate, lactose, etc.), binders (microcrystalline cellulose, starches, polyvinyl-pyrrolidone, etc.), disintegrating agents (starches and modified starches, cellulose derivatives, alginic derivatives, pectins, etc.), lubricants and flow enhancers (talc, magnesium stearate, colloidal silica, etc.), taste-masking agents (α-cyclodextrin, β-cyclodextrin, γ-cyclodextrin and derivatives thereof), flavorings or colorants, and also film-coating agents (for example: cellulose derivatives, methacrylic resins, polyvinyl chloride, nylons, etc.).

The pharmaceutical compositions according to the present invention can be provided in a solid and liquid form. It is important to underline that the beneficial effects of the invention should be observed, whatever the presentation of the galenic form. The pharmaceutical compositions according to the invention can be provided in the form of syrups, tablets, multilayer tablets, granules, microgranules, capsules, gelatin capsules, etc., these forms being coated or uncoated.

The pharmaceutical forms of capsule or gelatin capsule type may, for example, contain a mixture of immediate-release and prolonged-release granules. Prolonged-release granules can also be mixed with an immediate-release formulation and compressed into a tablet.

The pharmaceutical form can be provided, for example, as a transparent or opaque gelatin capsule containing two tablets, one of which contains the fraction which allows immediate release and the other of which contains the fraction which allows prolonged release (tablet which can be covered with a film-coating which allows the prolonged release).

Another presentation can envision gelatin capsules containing a mixture of microgranules, some of which may be covered with a film-coating which allows prolonged release of the active principle, and the others of which allow immediate release of the active principle.

The pharmaceutical form containing the combination according to the present invention is preferably provided as a double-layer or multilayer tablet, more particularly as tablets prepared by being subjected to more than one compression. The result of such a form may be either that of a tablet having two or more layers, or that of a tablet inside another tablet, the two parts releasing the active principle in a different way.

Preferably, when these two types of fractions are present in a two-layer tablet form, the pharmaceutical compositions which can be administered orally comprise:
A. at least one layer comprising the active principle and excipients which allow immediate release of said active principle after administration, and
B. at least a second layer which allows controlled release of the same active principle, comprising the active principle and at least one excipient which makes it possible to delay its release.

Such combined pharmaceutical compositions can be prepared according to various methods known to those skilled in the art.

More particularly, these combined pharmaceutical compositions can be provided in the form of a tablet in which at least one layer A is placed side by side with at least one layer B. In this case, such pharmaceutical compositions can be prepared by a process comprising the following successive steps:
1) preparing separate homogeneous mixtures from the components of layers A and B, and
2) compressing the homogeneous mixtures obtained in 1) in a multilayer tableting machine.

Optionally, the preparation of the homogeneous mixtures obtained in step 1) can contain a step consisting in granulating certain components.

The multilayer tableting machines for preparing this type of tablet are multilayer tableting machines of the Courtoy, Manesty, Hata, Fette, Killian type, etc.

The multilayer tablets are particularly suitable for implementing the present invention.

The research studies carried out in the development of novel pharmaceutical compositions which can be administered in a single daily dose have demonstrated a specific distribution of the active principle between the immediate-release fraction and the prolonged-release fraction. In fact, the, present invention relates most particularly to compositions comprising from 10 to 70 mg of efletirizine, in which the ratio of the amount of active principle in the immediate-release fraction to the amount of active principle in the controlled-release fraction is between 3 and 0.025, preferably between 1.6 and 0.05. The determination of the two equations, defined above, delimiting the ideal amounts efletirizine in each of the fractions of the composition as defined by the invention, was developed from the data presented in example 6.

Another unexpected and surprising advantage according to the present invention is that combining at least one immediate-release fraction and at least one controlled-release fraction makes it possible to limit or prevent the decrease in the maximum plasma concentration (Cₘₐₓ) and also limit or prevent modification of the bioavailability of the efletirizine, whether or not the patient has a meal before ingesting the medicinal product, the effectiveness of this system obviously being related to the distribution of the active principle between the fractions. Thus, the present invention relates most particularly to compositions comprising from 10 to 70 mg of efletirizine, in which the ratio of the amount of active principle in the immediate-release fraction to the amount of active principle in the controlled-release fraction is between 3 and 0.025. Maintenance of the maximum plasma concentration and of the bioavailability of the efletirizine is particularly observed for the novel pharmaceutical compositions corresponding to the equations given above.

The main pharmacokinetic parameters revealing that having a meal does not cause the Cₘₐₓ to decrease and that the bioavailability is not significantly modified for the compositions according to the invention are disclosed in examples 4, 5 and 6. Example 4 shows, for its part, that having a meal has a very marked influence on the pharmacokinetic parameters of an immediate-release composition: the Cₘₐₓ is decreased by 42%. The bioavailability is also decreased.

The examples which follow illustrate the present invention without, however, limiting it. Such pharmaceutical compositions combining an immediate-release fraction with a prolonged-release fraction can be prepared according to various conventional methods well known in the specialist literature.

The following abbreviations are used. PR: prolonged release; IR: immediate release; Cₘₐₓ: maximum plasma concentration; tₘₐₓ: the time taken to reach the Cₘₐₓ; AUC: area under the curve; the AUC characterizes the bioavailability and can be expressed in nanogram hour per milliliter or in % relative to the bioavailability of the reference solution.

### EXAMPLE 1

### Conventional Prolonged-Release (PR) Tablets Containing a Dose of 30 mg of Efletirizine

Nine formulations of tablets containing a dose of 30 mg of efletirizine were prepared and their composition is given in table 1. The efletirizine is granulated with one of more excipients and the other excipients (external phase) are added to this granulated material. The mixture is then compressed.

TABLE 1
Composition of the conventional PR tablets
(A to I)

| Components mg/tablet | A | B | C | F | G | H | I |
|---|---|---|---|---|---|---|---|
| Granulated material: | | | | | | | |
| Efletirizine | 30 | 30 | 30 | 30 | 30 | 30 | 30 |
| dihydrochloride | | | | | | | |
| Dibasic calcium | 70 | 145.5 | 97 | 35 | — | — | 59 |
| phosphate hydrate | | | | | | | |
| (Emcompress ™) | | | | | | | |
| Hydroxypropyl- | — | 45 | 25 | — | — | — | — |
| methylcellulose¹ | | | | | | | |
| (Methocel K15 MCR ™)² | | | | | | | |
| Beta-cyclodextrin | — | — | — | — | 82 | 82 | — |
| External phase: | | | | | | | |
| Hydroxypropylmethyl- | 160 | — | — | — | — | — | — |
| cellulose¹ | | | | | | | |
| (Methocel K100 | | | | | | | |
| MCR ™)³ | | | | | | | |
| Hydroxypropylmethyl- | — | 75 | 75 | 33.3 | 70 | — | — |
| cellulose¹ | | | | | | | |
| (Methocel K15 MCR ™)² | | | | | | | |
| Hydroxypropylmethyl- | — | — | — | — | — | 40 | 50 |
| cellulose¹ | | | | | | | |
| (Methocel E4 MCR ™)⁴ | | | | | | | |
| Microcrystalline | — | — | — | — | — | 45 | — |
| cellulose | | | | | | | |
| (Avicel pH 102 ™) | | | | | | | |
| Dibasic calcium | 134 | — | — | — | 15 | — | 13 |
| phosphate hydrate | | | | | | | |
| (Emcompress ™) | | | | | | | |
| Sodium bicarbonate | — | — | — | — | — | — | — |
| Magnesium stearate | 4 | 3 | 2 | 1 | 2 | 2 | 2 |
| Colloidal silica | 2 | 1.5 | 1 | 0.7 | 1 | 1 | 1 |
| (Aerosil 200 ™) | | | | | | | |

¹USP substitution type: 2208
²Nominal viscosity (EP method) = 7382 mPa · s
³Nominal viscosity (EP method) = 18243 mPa · s
⁴Nominal viscosity (EP method) = 3000 mPa · s (USP: HPMC 2910)

These tablets were the subject of 3 bioavailability studies on 8 volunteers taking again the tablets A, B and C (study A225), D (study A231) and E, F and G (study A244). Each time, the reference used is a solution of efletirizine administered at the same dose. The main pharmacokinetic parameters relating to these studies are given in tables 2 to 4.

TABLE 2
Main pharmacokinetic parameters of
bioavailability study A225

| Parameters | Solution | A | B | C |
|---|---|---|---|---|
| Cₘₐₓ (ng/ml) | 526 | 111 | 139 | 150 |
| tₘₐₓ (h) | 1 | 4 | 3.87 | 3.5 |
| AUC (ng · h/ml) | 2777 | 1603 | 1880 | 2159 |
| AUC (% solution) | 100 | 57.7 | 67.7 | 77.4 |

TABLE 3
Main pharmacokinetic parameters of
bioavailability study A231

| Parameters | Solution | D |
|---|---|---|
| Cₘₐₓ (ng/ml) | 561 | 158 |
| tₘₐₓ (h) | 0.88 | 3.52 |
| AUC (ng · h/ml) | 2891 | 2242 |
| AUC (% solution) | 100 | 77.6 |

TABLE 4
Main pharmacokinetic parameters of
bioavailability study A244

| Parameters | Solution | E | F | G |
|---|---|---|---|---|
| Cₘₐₓ (ng/ml) | 583 | 146 | 204 | 165 |
| tₘₐₓ (h) | 0.75 | 4 | 4 | 3.5 |
| AUC (ng · h/ml) | 2827 | 1717 | 1989 | 1775 |
| AUC (% solution) | 100 | 60.7 | 70.4 | 63 |

For all the PR forms, a Cₘₐₓ approximately 3 to 5 times lower than that corresponding to the reference solution can be noted. The AUC is also smaller: for the PR tablets, it reaches only 60 to 80% of that of the reference solution.

### EXAMPLE 2

### Administration of 2 Times 15 mg of Immediate-Release (IR) Efletirizine with a 12-hour Interval

Two times 15 mg of efletirizine in solution were administered to 12 healthy volunteers, with a 12-hour interval. The main pharmacokinetic parameters are given in table 5.

TABLE 5
Main pharmacokinetic parameters after administration
of 2 times 15 mg of efletirizine in solution,
with a 12-hour interval

| Parameter | |
|---|---|
| Cₘₐₓ(1) (ng/ml) | 307 |
| Cₘₐₓ (2) (ng/ml) | 305 |
| tₘₐₓ (1) (h) | 0.6 |
| tₘₐₓ (2) (h) | 12.6 |
| AUC (ng · h/ml) | 2800 |

### EXAMPLE 3

### Pharmacokinetic Simulations Based on the Results of Examples 1 and 2

Given the kinetics observed for the PR (example 1) and IR (example 2) forms and given the fact that it could be shown that it was necessary to combine an IR form with a PR form in order to obtain a sufficient Cₘₐₓ, simulations were carried out. These simulations related to doses to be administered in IR form (5 and 10 mg) and in PR form (20, 25 and 30 mg) of efletirizine in order to obtain a Cₘₐₓ and an AUC similar to those observed after administration of 2 times 15 mg with a 12-hour interval. The PR formulations chosen for these simulations were those which gave the best AUC values in example 1, i.e. the formulations C, D and F. The main kinetic parameters derived from these simulations are given in tables 6 to 10.

TABLE 6
Main pharmacokinetic parameters after simulations
using the kinetics observed in examples 1 and 2
as a basis
IR/PR doses: 5/25 mg

| | PR formulations | | |
|---|---|---|---|
| Parameters | C | D | F |
| Cₘₐₓ (ng/ml) | 186.2 | 187.5 | 219.4 |
| tₘₐₓ (h) | 3.0 | 2.0 | 3.0 |
| AUC (ng · h/ml) | 2201 | 2369 | 2083 |

TABLE 7
Main pharmacokinetic parameters after simulations
using the kinetics observed in examples 1 and 2 as a basis
IR/PR doses: 10/20 mg

| | PR formulations | | |
|---|---|---|---|
| Parameters | C | D | F |
| Cₘₐₓ (ng/ml) | 233.9 | 246.5 | 258.4 |
| tₘₐₓ (h) | 1.5 | 1.5 | 2.0 |
| AUC (ng · h/ml) | 2297 | 2404 | 2266 |

TABLE 8
Main pharmacokinetic parameters after
simulations using the kinetics observed in
examples 1 and 2 as a basis
IR/PR doses: 10/25 mg

| | PR formulations | | |
|---|---|---|---|
| Parameters | C | D | F |
| Cₘₐₓ (ng/ml) | 250.8 | 266.6 | 286.7 |
| tₘₐₓ (h) | 1.5 | 1.5 | 2.0 |
| AUC (ng · h/ml) | 2645 | 2788 | 2548 |

TABLE 9
Main pharmacokinetic parameters after
simulations using the kinetics observed in examples 1
and 2 as a basis
IR/PR doses: 5/30 mg

| | PR formulations | | |
|---|---|---|---|
| Parameters | C | D | F |
| Cₘₐₓ (ng/ml) | 211.9 | 210.4 | 251.7 |
| tₘₐₓ (h) | 3.0 | 2.0 | 3.0 |
| AUC (ng · h/ml) | 2553 | 2762 | 2407 |

TABLE 10
Main pharmacokinetic parameters after
simulations using the kinetics observed in examples 1
and 2 as a basis
IR/PR doses: 10/30 mg

| | PR formulations | | |
|---|---|---|---|
| Parameters | C | D | F |
| Cₘₐₓ (ng/ml) | 269.7 | 286.6 | 314.9 |
| tₘₐₓ (h) | 3.0 | 1.5 | 2.0 |
| AUC (ng · h/ml) | 2995 | 3175 | 2871 |

The best results, both for the Cₘₐₓ and for the AUC, are obtained for the IR/PR dosage of 10/25 mg, in particular using the PR formulation: D (table 8).

### EXAMPLE 4

### Administration of 60 mg of IR Efletirizine: Influence of a Meal

A gelatin capsule containing 60 mg of efletirizine was administered to 20 fasting patients and to 22 patients who had eaten a standardized fatty meal beforehand. The main pharmacokinetic parameters are given in table 11.

TABLE 11
Main pharmacokinetic parameters after
administration of 60 mg of efletirizine while fasting
or after a meal

| Parameters | Fasting | After a meal |
|---|---|---|
| Cₘₐₓ (ng/ml) | 1474 | 855 |
| tₘₐₓ (h) | 0.77 | 3.03 |
| AUC (ng · h/ml) | 6354 | 5784 |

It can be noted that having a meal has a very marked influence: the Cₘₐₓ is decreased by 42%. The bioavailability is also decreased.

### EXAMPLE 5

### Administration of 10 mg of IR Efletirizine Together with a PR Tablet Containing a Dose of 25 mg

In a crossover trial, 12 volunteers received 3 types of treatments:
10 mg of IR efletirizine in the form of a solution and one PR tablet of 25 mg of efletirizine (J) while fasting;
10 mg of IR efletirizine in the form of a solution and one PR tablet of 25 mg of efletirizine (J) after a standardized fatty meal;
15 mg of IR efletirizine in the form of a solution, taken twice with a 12-hour interval

The formula of the PR tablet containing 25 mg of active principle (J) used in this study is given in table 12. The first 3 components are granulated. The last 3 are then added and the mixture is compressed. The tablet J is a repeat of the composition of the tablet F, to which a granulating agent, Povidone K30™, has been added.

TABLE 12
Composition of the PR tablet containing a
dose of 25 mg of efletirizine (J)

| | Tablet J |
|---|---|
| Components | mg/tablet |
| Efletirizine dihyrochloride | 25 |
| Dibasic calcium phosphate | 30 |
| hydrate | |
| (Emcompress ™) | |
| Polyvinylpyrrolidone | 1.7 |
| (Povidone K30 ™) | |
| Hydroxypropylmethylcellulose | 28.4 |
| (Methocel K 15MCR ™) | |
| Colloidal silica | 0.6 |
| (Aerosil 200 ™) | |
| Magnesium stearate | 0.8 |

The main kinetic parameters are given in table 13.

TABLE 13
Main pharmacokinetic parameters after administration
of 10 mg of IR efletirizine and 25 mg of PR
efletirizine while fasting and after a meal, in
comparison with the administration of 2 times 15 mg of
IR efletirizine, with a 12-hour interval

IR/PR 10/25 mg

| Parameter | Fasting | After a meal | IR 15 mg × 2 |
|---|---|---|---|
| Cₘₐₓ (1) (ng/ml) | 280 | 355 | 307 |
| Cₘₐₓ (2) (ng/ml) | | | 305 |
| tₘₐₓ (1) (h) | 1.5 | 4 | 0.6 |
| tₘₐₓ (2) (h) | | | 12.6 |
| AUC (ng · h/ml) | 2680 | 3031 | 2801 |

Surprisingly, contrary to what had been observed in example 4, having a meal does not cause the Cₘₐₓ to decrease and the bioavailability is not significantly modified.

Also surprisingly, despite the absence of basifying agent in the PR formulation, the in vivo absorption proves to be constant and therefore independent of the pH, contrary to what had been observed in vitro in the dissolution tests described in patent application PCT/BE98/00033. This pH-independence results in small inter-individual variations after administration of the IR/PR form, and the coefficients of variation on the AUC are respectively 23% and 17% while fasting and after a meal.

### EXAMPLE 6

### Simulation of the Surface Areas Under the Curve for Combinations of IR and PR Forms of Efletirizine

Based on the pharmacokinetics obtained in example 5, simulations were performed for combinations at various concentrations of efletirizine in IR and PR forms. For each of these combinations (IR form from 0 to 20 mg and PR form from 15 to 35 mg), the surface area under the curve was calculated as a percentage relative to that obtained for the IR reference, 2 times 15 mg. The confidence limit at the threshold of 90% for these percentages was calculated and is 7%.

Compositions for which the surface areas under the curve expressed as a percentage, given a variability of 7%, are within the range of 80 to 125% can be considered to be bioequivalent.

Based on the data obtained, “modeling” taking again the various possible immediate- and delayed-release compositions bioequivalent to 2×15 mg of an immediate-release form made it possible to calculate the equations for two regression lines using the compositions directly below and above the confidence limits (80-125%).

The two lines obtained are defined by the following equations:
Y=−0.6786X+34 (R²=0.9918)
Y=−0.6636X+23.924 (R²=0.9955)
for which,
Y represents the amount of active principle in milligrams (mg) in the immediate-release fraction, and
X represents the amount of active principle in milligrams (mg) in the prolonged-release fraction,
R² represents the correlation coefficients for the two lines.

The values below the line for the equation

Y=−0.6636X+34 correspond to compositions whose bioavailability is too low, and those above the line for the equation Y=−0.6786X+23.924 correspond to compositions whose bioavailability is too high. The values included on or between the two lines, which are therefore bioequivalent to the administration of an immediate-release form of 2 times 15 mg IR, should therefore be taken into consideration.

Since the pharmacokinetics are linear, the equations for the lines delimiting the compositions bioequivalent to two administrations, with a 12-hour interval, of other doses, i.e. Z mg, become:
Y=−0.6786X+34Z/15 or Y=−0.6786X+2.267Z (Eq. 1)
Y=−0.6636X+23.924Z/15Y=−0.6636X+1.595Z (Eq. 2)

Z being between 5 and 25, and preferably between 7 and 15.

Thus, for Z=25, Eq. 1 becomes:
Y=−0.6786X+56.675
and for Z=5, Eq. 2 becomes:
Y=−0.6636X+7.975

### EXAMPLE 7

### Preparation Bioequivalent to 2 Administrations of 10 mg of Efletirizine in Immediate-Release Form, with a 12 h Interval

TABLE 14
Composition of a tablet containing a dose of
23.4 mg of efletirizine, bioequivalent to 2 administrations
of 10 mg of efletirizine in immediate-release
form, with a 12 h interval

| Components | mg/tablet |
|---|---|
| PR layer: | |
| Efletirizine dihydrochloride | 16.7 |
| Dibasic calcium phosphate hydrate | 30.0 |
| (Emcompress ™) | |
| Hydroxypropylmethylcellulose | 28.6 |
| (Methocel K15 MCR ™) | |
| Microcrystalline cellulose | 8.3 |
| (Avicel PH 101 ™) | |
| Colloidal silica (Aerosil 200 ™) | 0.6 |
| Magnesium stearate | 0.8 |
| IR layer: | |
| Efletirizine dihydrochloride | 6.7 |
| Lactose monohydrate | 45.6 |
| Microcrystalline cellulose | 26.5 |
| (Avicel PH 102 ™) | |
| Colloidal silica (Aerosil 200 ™) | 0.4 |
| Magnesium stearate | 0.8 |
| Coating: | |
| Hydroxypropylmethylcellulose + titanium | 4.95 |
| dioxide | |
| (White Opadry Y-1-7000 ™) | |

### EXAMPLE 8

### Preparation Bioequivalent to 2 Administrations of 7 mg of Efletirizine in Immediate-Release Form, with a 12 h Interval

TABLE 15
Composition of a tablet containing a dose of
16.4 mg of efletirizine, bioequivalent to 2 administrations
of 7 mg of efletirizine in immediate-release
form, with a 12 h interval

| Components | mg/tablet |
|---|---|
| PR layer: | |
| Efletirizine dihydrochloride | 11.7 |
| Dibasic calcium phosphate hydrate | 30.0 |
| (Emcompress ™) | |
| Hydroxypropylmethylcellulose | 28.6 |
| (Methocel K15 MCR ™) | |
| Microcrystalline cellulose | 13.3 |
| (Avicel PH 101 ™) | |
| Colloidal silica (Aerosil 200 ™) | 0.6 |
| Magnesium stearate | 0.8 |
| IR layer: | |
| Efletirizine dihydrochloride | 4.7 |
| Lactose monohydrate | 46.6 |
| Monocrystalline cellulose | 27.5 |
| (Avicel PH 102 ™) | |
| Colloidal silica (Aerosil 200 ™) | 0.4 |
| Magnesium stearate | 0.8 |
| Coating: | |
| Hydroxypropylmethylcellulose + titanium | 4.95 |
| dioxide | |
| (White Opadry Y-1-7000 ™) | |

1. A pharmaceutical composition which can be administered orally, containing efletirizine as active principle, wherein it combines at least one fraction which allows the immediate release of the efletirizine and at least one fraction which allows prolonged release of efletirizine.

2. The composition as claimed in claim 1, wherein the total amount of efletirizine in the composition is between 10 and 70 mg, and the weight ratio of the amount of active principle in the immediate-release fraction to the amount of active principle in the prolonged-release fraction is between 3 and 0.025.

3. A pharmaceutical composition which can be administered orally, containing efletirizine as active principle, wherein it combines at least one fraction which allows immediate release of the efletirizine and at least one fraction which allows prolonged release of the efletirizine, the respective amounts of active principle in the two fractions being the values included on or between the two straight lines defined by the following equations:
Y=−0.6786X+56.675
Y=−0.6636X+7.975
in which,
Y represents the amount of efletirizine in milligrams (mg) in the immediate-release fraction, and
X represents the amount of efletirizine in milligrams (mg) in the prolonged-release fraction, and
the total amount X+Y being between 10 and 70 mg.

4. The composition as claimed in claim 3, wherein it can be administered in a single daily dose, while obtaining the desired therapeutic effect.

5. The composition as claimed in claim 1, wherein the two fractions are provided in the form of a two-layer tablet.

6. The composition as claimed in claim 1, wherein the fraction which allows prolonged release of the efletirizine contains less than 5% by weight of basifying agent, weight relative to the total weight of the fraction.
